(19) United States
(12) Patent Application Publication
(10) Pub. No.: US 2009/0093729 A1
(43) Pub. Date: Apr. 9, 2009

(54) Devices and Methods for Measurement of Vessel Elasticity and Blood Pressure

(76) Inventors: Yunlong Zhang, Mounds View, MN (US); Bin Mi, Plymouth, MN (US); Yongxing Zhang, Maple Grove, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(21) Appl. No.: 11/866,523

(22) Filed: Oct. 3, 2007

(51) Int. Cl. A61B 5/0215

(52) U.S. Cl. 600/486

(57) ABSTRACT

Embodiments of the invention are related to devices and methods for measuring arterial elasticity and/or blood pressure, amongst other things. In an embodiment, the invention includes an implantable medical device having a sensor element that is configured to engage a vessel of a patient. The sensor element is further configured to generate a signal in response to bending of the sensor element, where bending occurs as a result of changes in the pressure within the vessel. The implantable medical device further includes a controller in signal communication with the sensor element, where the controller is configured to store information regarding the signal generated by the sensor element. Other embodiments are also included herein.

### TECHNICAL FIELD

This disclosure relates generally to implantable devices and methods for measuring arterial elasticity and blood pressure.

### BACKGROUND OF THE INVENTION

Hypertension, or elevated blood pressure, is a common cardiovascular condition afflicting greater than 50 million people by some estimates. Hypertension is a serious risk factor associated with stroke and cardiovascular morbidity. It is believed to directly contribute to at least 250,000 deaths per year.

Frequent measurement of blood pressure is important for monitoring the health of hypertensive patients and for the titration of blood pressure lowering medications. Typically, blood pressure is measured using the auscultatory method. The auscultatory method involves using a stethoscope and a sphygmomanometer. In brief, an inflatable cuff is placed around the upper arm and inflated until the artery is completely occluded. The pressure in the cuff is then slowly released and a stethoscope is used to listen for Korotkoff sounds. The auscultatory method is relatively easy to perform. However, it usually cannot be performed accurately by the patient themselves. As such, the frequency of blood pressure measurements taken by this method is generally limited to times when another individual can perform the method on the patient. External digital blood pressure monitoring systems have been developed to facilitate self measurement by the patient. However, patient compliance remains a significant issue with these systems.

In some cases, a catheter is used to assess blood pressure intravascularly. For example, a catheter including a pressure sensor can be inserted into the vasculature of a patient and blood pressure can be monitored directly. This method can provide highly accurate blood pressure measurements. Unfortunately, the intravascular placement of equipment creates risks of complications such as thromboembolism.

For at least these reasons, a need remains for devices and methods for measuring arterial elasticity and blood pressure.

### SUMMARY OF THE INVENTION

Embodiments of the invention are related to devices and methods for measuring arterial elasticity and/or blood pressure. In an embodiment, the invention includes an implantable medical device having a sensor element that is configured to engage a vessel of a patient. The sensor element is further configured to generate a signal in response to bending of the sensor element, where bending occurs as a result of changes in the pressure within the vessel. The implantable medical device further includes a controller in signal communication with the sensor element, where the controller is configured to store information regarding the signal generated by the sensor element.

Another embodiment relates to a method of measuring arterial elasticity and blood pressure. The method includes chronically implanting a sensor around the exterior of an artery of a patient. The sensor includes a sensor element that is configured to generate an electrical signal in response to bending of the sensor element. The method further includes processing the electrical signal from the sensor element to derive arterial elasticity and blood pressure.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a perspective view of a blood pressure sensor engaged with a vessel of a patient in accordance with an embodiment of the invention.

FIG. 2 is a cross-sectional view of an embodiment of a blood pressure sensor engaged with a vessel of a patient as taken along line 2-2′ of FIG. 1.

FIG. 3 is a top schematic view of an exemplary flexion sensor.

FIG. 4 a cross-sectional view of a flexion sensor as taken along line 4-4′ of FIG. 3.

FIG. 5 is a cross-sectional view of an alternate embodiment of a blood pressure sensor.

FIG. 6 is a cross-sectional view of an alternate embodiment of a blood pressure sensor.

FIG. 7 is a side view of an embodiment of a blood pressure sensor.

FIG. 8 is a side view of an alternate embodiment of a blood pressure sensor.

FIG. 9 is cross-sectional view of an embodiment of a blood pressure sensor having multiple sensor elements.

FIG. 10 is a side view of an embodiment of a blood pressure sensor having multiple sensor elements.

FIG. 11 is a perspective view of an embodiment of a blood pressure sensor having a plurality of retaining clip elements.

FIG. 12 is a cross-sectional view of an alternate embodiment of a blood pressure sensor.

FIG. 13 is a schematic depiction of functional elements of a controller device.

FIG. 14 is an embodiment of a medical system having a monitoring device in signal communication with a blood pressure sensor.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Measurement of blood pressure with an extravascular implantable device, in comparison to an intravascular device, can provide accurate pressure measurements while decreasing risks associated with intravascular devices, such as the risk of thromboembolism. In some embodiments, the invention can include extravascular devices for measuring blood pressure. For example, an embodiment can include an implantable medical device including a sensor element configured to engage the exterior surface of a vessel of a patient and change in diameter in response to changes in pressure within the vessel. The sensor element is configured to generate a signal in response to bending of the sensor element occurring as a result of changes in the diameter of the vessel. In some embodiments, a sensor housing is provided over the sensor element. Various aspects of devices in accordance with embodiments included herein will now be described in greater detail.

Referring now to FIG. 1, a perspective view of a blood pressure sensor 20 is shown in accordance with an embodiment of the invention. Blood pressure sensor 20 is shown engaged with an exterior surface 24 of a blood vessel 22 of a patient. Blood pressure sensor 20 is shown having a housing 28. A conductor 12, such as an electrical or an optical conductor, can provide signal communication between the blood pressure sensor 20 and other components. However, in some embodiments, the blood pressure sensor 20 is in wireless communication with other components.

Vessel 22 can specifically include, but is not limited to, arteries such as the ascending aorta, aortic arch, descending aorta, coronary arteries, brachiocephalic artery, common carotid artery, subclavian artery, internal carotid artery, external carotid artery, axillary artery, brachial artery, celiac trunk, left gastric artery, splenic artery, common hepatic artery, superior mesenteric artery, renal artery, gonadal artery, inferior mesenteric artery, lumbar arteries, common iliac artery, external iliac artery, femoral artery, deep femoral artery, and internal iliac artery. While not intending to be bound by theory, it is believed that positioning of a blood pressure sensor such as those included herein around a peripheral artery, such as the brachial artery, can be advantageous because the process of implanting the blood pressure sensor can be less invasive than implanting such a sensor around central arteries. As such, in some embodiments, the vessel 22 is a peripheral artery.

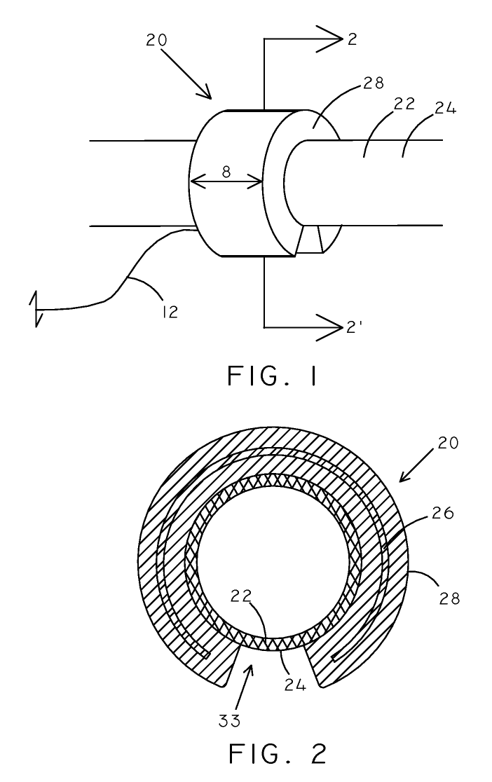

A cross-sectional view of blood pressure sensor 20 is shown in FIG. 2, as taken along line 2-2′ of FIG. 1. Vessel 22 is shown in cross-section and with blood pressure sensor 20 engaged around exterior surface 24. Blood pressure sensor 20 has a sensor element 26 that is within a housing 28. Both sensor element 26 and housing 28 can be constructed from a flexible, pliable material that is able to conform to the outer diameter of the vessel.

As blood pressure in the vessel varies, such as between systolic and diastolic pressure, the blood vessel will tend to expand and contract accordingly. The housing 28 and sensor element 26 within housing 28 have sufficient flexibility to expand and contract along with the vessel. Housing 28 and sensor element 26 may provide some resistance to expansion of vessel 22, but generally this resistance is minimal and does not prevent vessel 22 from expanding normally as pressure increases within vessel 22. In some embodiments, housing 28 has a width 8 (shown in FIG. 1) of less than about 1 mm.

Various types of sensor elements can be used with embodiments of the invention. By way of example, the sensor element can be a flexion sensor. In an embodiment, sensor element 26 is a flexion sensor that is configured to generate a signal in proportion to the pressure within the vessel to which it is engaged. The flexion sensor can be configured to generate a signal in response to flexion, including an electrical and/or an optical signal. The signal can then be processed in order to derive arterial elasticity and/or blood pressure.

In some embodiments, a flexion sensor generates an optical signal in response to flexion. In other embodiments, a flexion sensor generates an electrical signal in response to flexion. The electrical signal can be in the form of a current, a voltage, a resistance, or changes in the same. For example, the flexion sensor can be configured so that its resistance varies as a function of bending of the sensor. An exemplary electrical flexion sensor is described in U.S. Pat. No. 5,583,476, the content of which is herein incorporated by reference. An exemplary electrical flexion sensor is commercially available from Flexpoint Sensor Systems, Inc., Draper, Utah. In other embodiments, the flexion sensor can include a piezoelectric element that generates a current in response to bending of the flexion sensor. In an embodiment, a piezoelectric element is constructed from polyvinylidene fluoride.

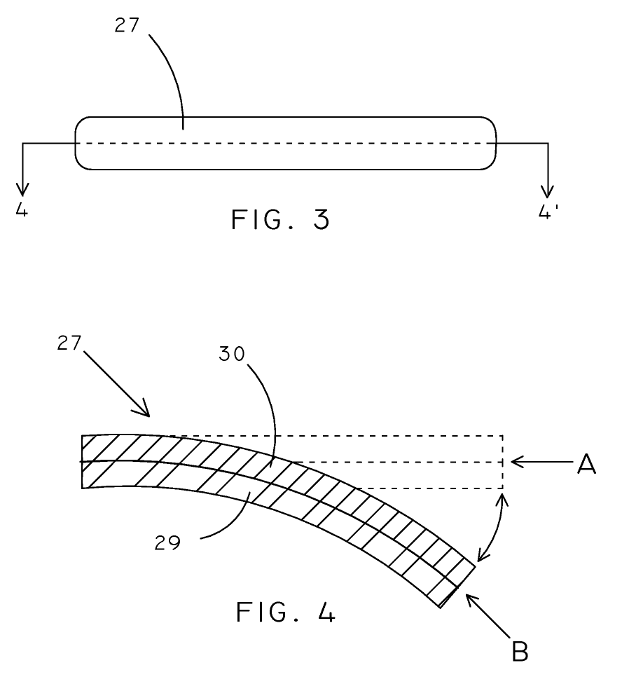

Referring now to FIG. 3, a top schematic view is shown of an exemplary flexion sensor 27 in accordance with an embodiment of the invention. FIG. 4 shows a cross-sectional view of the flexion sensor 27 as taken along line 4-4′ of FIG. 3. The flexion sensor 27 includes a substrate 29 and a conductive material layer 30 disposed over the substrate 29. In some embodiments, the substrate 29 is made from a flexible material, such as a polyamide, or another type of polymer. The conductive material layer 30 can be configured so that its resistance changes with the degree to which the conductive material layer 30 is flexed. For example, the flexion sensor 27 can assume a first position A and then flex in order to assume a second position B. The resistance of the conductive material layer 30 changes in a manner so as to allow the degree of flexion to be calculated. The conductive material layer 30 can include graphite in combination with a binder.

In some embodiments, flexion sensors used in devices and systems herein can be optical flexion sensors. Optical flexion sensors can include an optical conductor such as an optical fiber. Optical fibers generally include a core surrounded by a cladding layer. To confine the optical signal to the core, the refractive index of the core is typically greater than that of the cladding. The boundary between the core and cladding may either be abrupt, as in step-index fiber, or gradual, as in graded-index fiber. Optical signals can pass through the core of the optical fiber by means of total internal reflection. However, if the angle of incidence of light striking the boundary between the core and cladding exceeds a critical value, then some amount of the optical signal will pass outside of the optical fiber and not be reflected internally. As such, an optical fiber that is bent beyond a critical angle will exhibit some degree of optical signal loss. Therefore, bending of an optical fiber can be detected by monitoring the optical signals transmitted by the optical fiber. In one approach, an optical signal, such as that generated by a light emitting diode (LED), can be passed through an optical conductor, reflected at the tip, passed back through the optical conductor and then received by a component such as a photodiode.

In some embodiments, the optical conductor includes an unmodified optical fiber. However, in some embodiments, portions of the optical fiber cladding can be removed to enhance sensitivity of specific regions of the optical fiber to bending signal loss. In some embodiments, the optical conductor includes a bend-enhanced fiber (BEF) sensor. BEFs can be made by treating optical fibers to have an optically absorptive zone along a thin axial stripe. Light transmission through the fiber past this zone then becomes a robust function of curvature that is more sensitive to bending than otherwise similar untreated optical fiber.

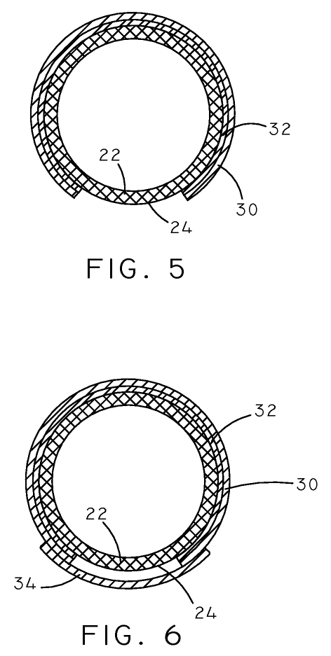

There are numerous configurations in which blood pressure sensor 20 can be oriented in relation to a vessel 22. In some embodiments, blood pressure sensor 20 has a generally “C”-shaped configuration that is partly wrapped or clipped around vessel 20. This configuration may also be referred to as a discontinuous annular configuration. An embodiment of this configuration is shown in FIGS. 1 and 2. The generally “C”-shaped configuration defines a gap 33. In some embodiments, such as FIG. 2, housing 28 encloses sensor element 26 such that housing 28 provides the surface that contacts exterior surface 24 of vessel 22 as well as providing an exposed surface that faces away from the vessel. In other embodiments, such as FIGS. 5 and 6, housing 30 only covers the surface of sensor element 32 that is not in contact with exterior surface 24 of vessel 22. In the embodiment of FIG. 6, a latch member 34 is attached across the gap 33 in housing 30 in order to provide additional support to housing 30 and to help prevent housing 30 from becoming detached from the vessel or shifting in location.

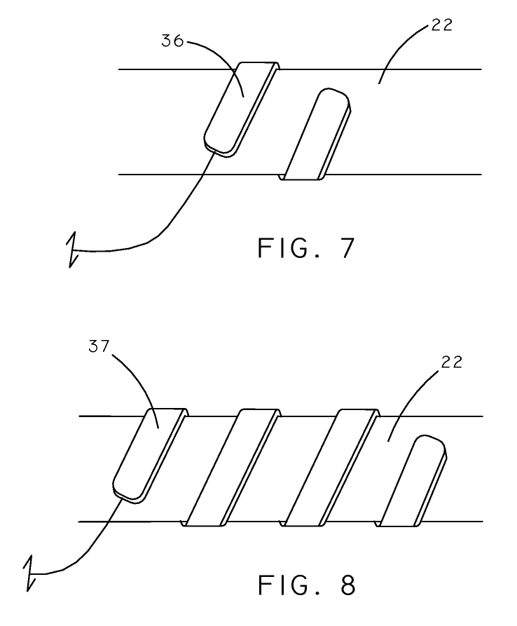

Referring now to FIG. 7, in another embodiment, blood pressure sensor 36 is wrapped in a helical configuration around vessel 22. This may also be referred to as a corkscrew configuration. The blood pressure sensor 36 can be wrapped around the vessel 22 such that it makes a relatively small number of turns around vessel 22, such as in the range of approximately two-thirds of a turn around vessel 22 to approximately one to one and a half turns around vessel 22, as shown in FIG. 7. In another embodiment, blood pressure sensor 36 can be wrapped around vessel 22 in a manner that it makes multiple turns around vessel 22, as shown in FIG. 8. For example, as shown in FIG. 8, sensor 36 makes approximately three and a quarter turns around vessel 22.

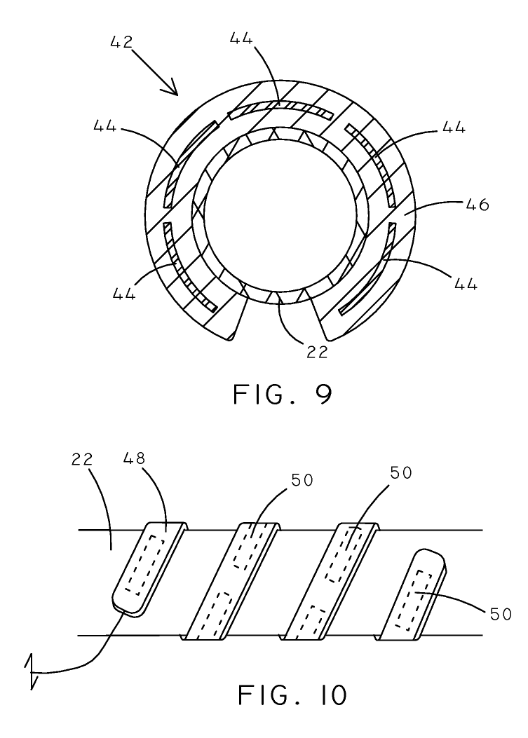

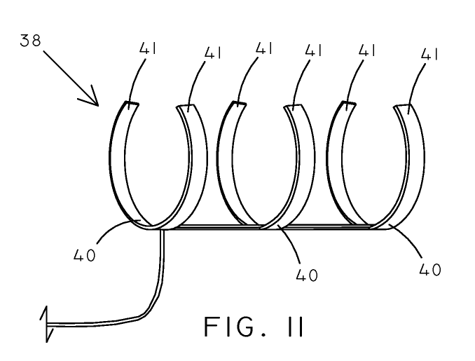

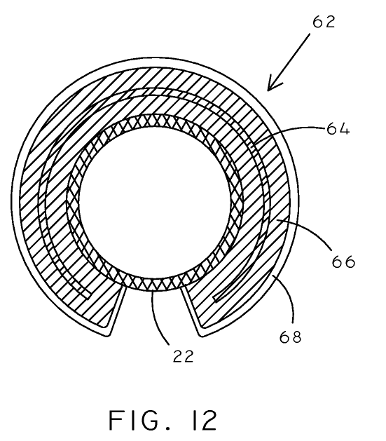

In some embodiments, more than one sensor element is provided with the blood pressure sensor. One advantage of such an arrangement is potentially improved accuracy. If the blood vessel does not expand uniformly around its circumference with an increase in pressure, the presence of a plurality of sensor elements can account for this non-uniformity. Further, where multiple sensor elements are included, the resulting multiple signals can be cross-referenced to reduce signal noise. Exemplary embodiments having a plurality of sensor elements are shown in FIGS. 9 and 10. As shown in cross-section in FIG. 9, blood pressure sensor 42 can have a plurality of sensor elements 44 arranged around the circumference of vessel 22 within a housing 46.

As shown in FIG. 10, a blood pressure sensor 48 has a plurality of sensor elements 50 arranged at intervals within a helical configuration and within a housing 52. In this configuration, the sensor elements are disposed at different positions along the lengthwise axis of the vessel 22. Thus, when a pressure wave passes through the vessel, such as when the heart contracts, those sensor elements on the upstream side of the vessel will generate a signal in response to expansion of the vessel just before those elements on the downstream side of the vessel. As such, the velocity of the pressure wave passing through the vessel can be measured. This velocity can be indicative of various cardiovascular parameters such as cardiac contractility, flow rate, and/or cardiac output.

In yet another embodiment, as shown in FIG. 11, a blood pressure sensor 38 may be constructed from a plurality of retaining clip elements 40. Each of retaining clip elements 40 can include a sensor element and are configured to engage with a vessel. In some embodiments, the clip elements can have a C-shaped configuration. Each clip element 40 can have a pair of arms 41 or appendages that engage the vessel.

When a foreign object, such as a medical device, is implanted within a patient, the patient's body reacts immunologically leading to the formation of fibrous tissue (sometimes referred to as a capsule) over the foreign object. This fibrous tissue can serve to interfere with the functions of some types of devices. In some cases, tissue in-growth may impair a blood pressure sensor's sensitivity and accuracy by constraining free and flexible expansion of the sensor as well as the expansion of the blood vessel to which the sensor is engaged.

Some embodiments of the invention can include extravascular implantable devices for measuring blood pressure that are configured to reduce issues associated with the growth of tissue. In some embodiments of the invention, the size of the device is kept to a minimum such that there is a relatively small foreign body response resulting in limited tissue in-growth. For example, in some embodiments, the width of the sensor housing and/or sensor element can be less than about 1 mm.

In some embodiments, a layer of a material is disposed on the sensor and/or the housing that serves to prevent tissue in-growth. Referring now to FIG. 12, a cross-sectional view of an alternate embodiment of a blood pressure sensor 62 is shown engaged with a vessel 22. The blood pressure sensor 62 can include a sensor element 64 and a housing 66. A tissue in-growth prevention layer 68 is disposed over the housing 66. In some embodiments, the tissue in-growth prevention layer 68 is a layer of a material such as polytetrafluoroethylene (PTFE). In other embodiments, the tissue in-growth prevention layer 68 is a drug elution layer configured to release tissue growth inhibiting drugs to prevent the sensor and/or housing from being substantially overgrown with tissue.

In some embodiments, the blood pressure sensor is pre-shaped, prior to implant, such as in a helical configuration or a C-shaped configuration. Such a blood pressure sensor is then screwed or wrapped around the vessel during implantation. In some embodiments, a housing can be provided that is snapped over the blood pressure sensor after the blood pressure sensor is installed on the vessel.

Embodiments of the invention can include a controller configured to receive signals from a sensor element and to perform various processes on the received signals. The controller can be implanted or can be external to a patient. In some embodiments, the controller is co-located with the sensor element. In other embodiments, the controller is located remotely from the sensor element, but in communication with the sensor element such as through a lead. For example, the controller can be part of an implanted cardiac rhythm management (CRM) device that is in communication with a sensor element. In some embodiments, more than one controller is present. For example, a first controller can be co-located with a sensor element and a second controller can be co-located with separate device. In some embodiments, functionalities of a controller are split across multiple physical components.

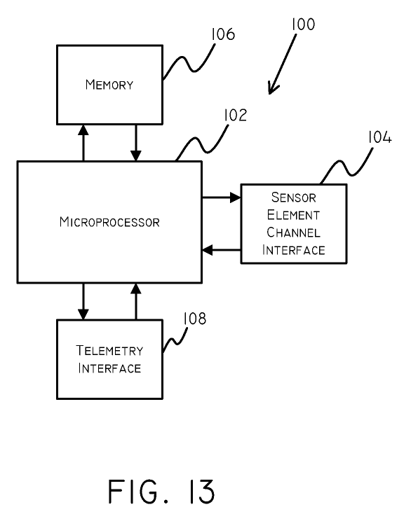

Referring now to FIG. 13, some components of an exemplary controller 100 are schematically illustrated. The controller 100 can include a microprocessor 102 that communicates with a memory 106 via a bidirectional data bus. The memory 106 typically comprises ROM or RAM for program storage and RAM for data storage. The controller 100 can be configured to execute various operations such as processing of signals and execution of methods as described herein. In some embodiments, such as where one or more components of the system operate wirelessly, a telemetry interface 108 can also be provided for communicating with other units, such as a programmer device, a patient management system, or another implanted device. Wireless communication can be conducted via radiofrequency, inductively, or acoustically. The controller 100 further includes a sensor element channel interface 104 which communicates bidirectionally with a port of microprocessor 102. The channel interface 104 can include an analog-to-digital converter for digitizing sensing signal inputs and other electronic components as necessary to interface with a blood pressure sensor.

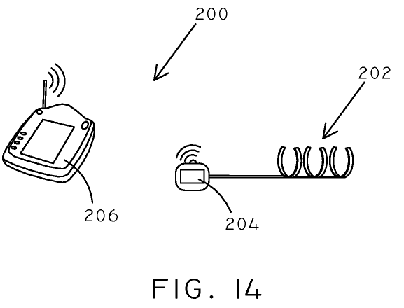

FIG. 14 shows an embodiment of a blood pressure sensor 202 incorporated into a medical device system 200. Generally, blood pressure sensor 202 is in signal communication with a controller 204 that is configured to receive the signals from the blood pressure sensor 202. Controller 204 may, in various embodiments, be configured to store, record, and/or process the signal received from the blood pressure sensor 202.

The controller 204 can be in wireless signal communication with an external device 206. An example of an external device is a patient management device such as the Latitude device available from Boston Scientific, Natick, Mass. Another example of an external device is a device programmer.

In some embodiments, the controller 204 can be part of a cardiac rhythm management device. Implantable medical devices can specifically include pacemakers, implantable cardioverter-defibrillators (ICDs), cardiac resynchronization therapy (CRT) devices, and the like.

In various embodiments herein, a signal from a sensor element of a blood pressure sensor can be processed in order to derive information regarding the patient's blood pressure. In order to determine the patient's blood pressure, it is often necessary to perform a calibration of the sensor readings to a known blood pressure. The calibration can be done on each patient by temporarily providing an additional blood pressure sensor of known calibration and comparing the readings against the signal from the implantable blood pressure sensor. Alternatively, statistical correlations may be developed on a representative patient or patients and may be used to define calibration parameters that allow the signal from a blood pressure sensor to be converted to a blood pressure value.

Embodiments of the invention can include various methods that include or rely upon data gathered with the aid of a flexion sensor. Such methods can be usefully applied in many contexts, including in the context of implantable medical devices that deliver a therapy based on the patient's blood pressure or that provide information to a person such as a physician regarding a patient's blood pressure. In some embodiments, a controller may analyze the signal from the blood pressure to determine whether a medical condition exists within the patient or whether a course of action is recommended.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. An implantable medical device comprising:
a sensor element configured to engage an outside surface of a vessel of a patient and to generate a signal in response to bending of the sensor element occurring as a result of changes in the pressure within the vessel; and
a controller in signal communication with the sensor element configured to store information regarding the signal generated by the sensor element.

2. The implantable medical device of claim 1, comprising a helical shape and extending greater than one complete turn around a lengthwise axis of the vessel.

3. The implantable medical device of claim 1, comprising a plurality of sensor elements configured to be positioned at different points radially around the exterior surface of the vessel.

4. The implantable medical device of claim 1, comprising a plurality of sensor elements configured to be positioned at different points along a lengthwise axis of the vessel.

5. The implantable medical device of claim 1, the controller configured to process the signal generated by the sensor element to determine the pressure of a fluid within the vessel.

6. The implantable medical device of claim 1, the sensor element comprising a C-shaped configuration.

7. The implantable medical device of claim 6, further comprising a latch member configured to retain the sensor element around the vessel.

8. The implantable medical device of claim 1, further comprising a sensor housing, the sensor housing configured to cover at least a portion of the sensor element.

9. The implantable medical device of claim 8, the surface of the sensor housing configured to reduce tissue in-growth.

10. The implantable medical device of claim 8, the sensor housing comprising a layer of material configured to release an active agent in vivo to reduce tissue in-growth.

11. The implantable medical device of claim 8, further comprising a layer of polytetrafluoroethylene disposed over the sensor housing.

12. The implantable medical device of claim 8, the sensor housing surrounding the sensor element.

13. The implantable medical device of claim 1, comprising a plurality of retaining clip elements, each retaining clip element configured to be disposed at a different position along the lengthwise axis of the vessel, each retaining clip element comprising a sensor element.

14. The implantable medical device of claim 1, the sensor element configured to generate an electrical signal in response to bending of the sensor element.

15. The implantable medical device of claim 14, the sensor element comprising a piezoelectric material.

16. The implantable medical device of claim 15, the piezoelectric material comprising polyvinylidene fluoride.

17. The implantable medical device of claim 14, the sensor element configured so that its electrical resistance varies as a function of bending.

18. The implantable medical device of claim 1, wherein the controller is in wireless signal communication with an external device.

19. A method of measuring blood pressure comprising:
processing an electrical signal from a sensor to derive blood pressure, the sensor chronically implanted around the exterior of an artery of a patient, the sensor comprising a sensor element configured to generate the electrical signal in response to bending of the sensor element.

20. The method of claim 19, comprising chronically implanting a sensor around the exterior of a peripheral artery of a patient.

21. The method of claim 19, comprising chronically implanting a sensor around the brachial artery of a patient.
